(19) United States
(12) Patent Application Publication
(10) Pub. No.: US 2015/0126864 A1
(43) Pub. Date: May 7, 2015

(54) IMAGE GENERATION APPARATUS

(71) Applicant: KONINKLIJKE PHILIPS N.V., EINDHOVEN (NL)

(72) Inventors: Thomas Buelow, Grosshansdorf (DE); Jörg Sabczynski, Norderstedt (DE); Kirsten Regina Meetz, Hamburg (DE)

(21) Appl. No.: 14/397,708

(22) PCT Filed: May 15, 2013
(86) PCT No.: PCT/IB2013/053967

§ 371 Date: Oct. 29, 2014

Related U.S. Application Data
(60) Provisional application No. 61/651,062, filed on May 24, 2012.

(51) Int. Cl. A61B 8/00; A61B 8/08

(52) U.S. Cl. CPC A61B 8/4254; A61B 8/0825; A61B 8/5253; A61B 8/5207; A61B 8/54; A61B 8/40
USPC 600/437

(57) ABSTRACT

The invention relates to an image generation apparatus (1) for generating an image of a deformable object (5) like a woman's breast. A first image generating unit (2) like an ultrasound head generates a first image of the object covered by a flexible cover element (6), wherein a shape sensing and localization unit (10), in particular, a fiber-optic shape sensing and localization unit, determines the shape and position of the cover element. The apparatus further comprises a position determination unit (11) for determining the position of the first image generating unit relative to the cover element. Information about the deformation of the deformable object with respect to the first image can therefore be provided, which can facilitate reviewing the first image, especially reviewing of breast images. For instance, this information can facilitate a comparison of breast images acquired by different imaging modalities.

### FIELD OF THE INVENTION

The invention relates to an image generation apparatus, an image generation method and an image generation computer program for generating an image of a deformable object. The invention relates further to a holding device for holding a woman's breast during a generation of an image of the breast. The invention relates also to a registration device, a registration method and a registration computer program for registering images with respect to each other.

### BACKGROUND OF THE INVENTION

During ultrasound imaging of a woman's breast an ultrasound head of an ultrasound imaging device is in contact with the breast and deforms the same. The generated ultrasound image is therefore an image of a deformed breast. The deformation of the breast can render reviewing of the ultrasound image by a radiologist, in particular, a comparison of the ultrasound image showing the deformed breast with other images like images acquired by the same ultrasound imaging device with the ultrasound head at another location on the deformed breast and/or in another orientation or like images of other imaging modalities very difficult. For instance, the deformation of the breast, which is generally different in different images, renders a finding of anatomically corresponding locations in the different images difficult.

### SUMMARY OF THE INVENTION

It is an object of the present invention to provide an image generation apparatus, an image generation method and an image generation computer program for generating an image of a deformable object, which facilitate a reviewing of the image of the deformable object, in particular, a comparison of the image with an image of the same object generated by another imaging modality. It is a further object of the present invention to provide a holding device for holding a woman's breast, which allows for a facilitated reviewing of an image of the breast generated while the woman was wearing the holding device, in particular, for a facilitated comparison of the generated image with another image of the breast generated by another imaging modality. Moreover, it is an object of the present invention to provide a registration device, a registration method and a registration computer program for registering two images such that corresponding regions of the object in the different images can reliably be determined, even if the object is deformed in at least one of the images.

In a first aspect of the present invention an image generation apparatus for generating an image of a deformable object is presented, wherein the image generation apparatus comprises:
a first image generating unit for generating a first image of the object,
a flexible cover element for covering the object,
a shape sensing and localization unit for determining the shape and position of the cover element, wherein the determined shape and position of the cover element are the shape and position during the generation of the first image,
a position determination unit for determining the position of the first image generating unit relative to the cover element, wherein the determined position of the first image generating unit relative to the cover element is the position during the generation of the first image of the object.

Since the deformable object, which is preferentially a woman's breast, is covered by the flexible cover element, wherein the shape and position of the cover element are determined during the generation of the first image and since during the generation of the first image also the position of the first image generating unit, which is preferentially an ultrasound head, relative to the cover element is determined, information is provided, which can facilitate reviewing the first image. For instance, since the flexible cover element covers the deformable object and since the shape and position of the cover element have been determined, the deformation of the deformable object is known. Moreover, since the position of the first image generating unit is known relative to the cover element, the deformation of the deformable object is known with respect to the first image, i.e. in a coordinate system of a first image space in which the first image is defined. This knowledge about the deformation of the deformable object with respect to the first image, i.e. this additional deformation information, can facilitate the reviewing of the first image.

For instance, the image generation apparatus may further comprise a second image providing unit for providing a second image of the object, a surface determination unit for determining a surface of the object in the second image, and a registration unit for registering the first image and the second image with respect to each other based on i) the determined shape and position of the cover element covering the object, ii) the determined position of the first image generating unit relative to the cover element and iii) the determined surface of the object in the second image. The knowledge about the shape and position of the cover element, and hence about the outer surface of the deformed object, relative to the first image can be used to register the first image with the second image in which the outer surface of the deformable object is detectable, for instance, by using known segmentation algorithms, wherein for registering the first image and the second image with respect to each other, the determined shape and position of the cover element can be registered with the outer surface of the object determined in the second image.

The first image generating unit is preferentially a first imaging modality, wherein the second image is an image of a second imaging modality being different to the first imaging modality. In particular, the first image can be a two-dimensional image and the second image can be a three-dimensional image. For instance, the first image can be a two-dimensional ultrasound image of the object and the second image can be a three-dimensional magnetic resonance image of the object. The first image generating unit is, for example, an ultrasound head, which may be pressed against a woman's breast, for generating an ultrasound image of the breast, wherein in the second image the deformable object is preferentially substantially not compressed, i.e. it may only be deformed a little by gravity.

The second image providing unit can be a storing unit, in which the second image is stored already and from which the second image can be retrieved for providing the same, or the second image providing unit can be a receiving unit for receiving the second image from an imaging modality like a magnetic resonance imaging system, wherein the second image providing unit can be adapted to provide the received second image. The second image providing unit can also be an imaging modality generating the second image and providing the generated second image.

The flexible cover element comprises preferentially a shape sensing element for generating a signal depending on its shape, wherein the shape sensing and localization unit is connected to the shape sensing element and adapted to determine the shape and position of the cover element based on the signal from the shape sensing element. The shape sensing element is preferentially an optical fiber for fiber-optic shape sensing and localization (FOSSL), wherein the shape sensing and localization unit is preferentially a fiber-optic shape sensing and localization unit being connected to the optical fiber for receiving an optical signal from the optical fiber and being adapted to determine the shape and position of the cover element by FOSSL from the optical signal using the optical fiber.

The cover element is preferentially a membrane, in particular, a polymeric membrane. The cover element may comprise one or several shape sensing elements, which are connected to the shape sensing and localization unit and which are interwoven in the cover element. In a preferred embodiment the shape sensing elements are arranged in parallel or in a rectangular grid in the cover element, wherein neighboring shape sensing elements can have a regular spacing of, for instance, 5 mm. If the shape sensing elements are arranged in this way, the accuracy of determining the shape and position of the cover element and, thus, of the outer surface of the deformable object can be further improved.

It is preferred that the shape sensing and localization unit is adapted to determine the shape and the position of the cover element by determining the shape and the position of the shape sensing element of the cover element and by reconstructing a surface defined by the cover element based on the determined shape and position of the shape sensing element, thereby determining the shape and position of the cover element. For instance, the cover element can comprise several shape sensing elements, wherein a three-dimensional interpolation algorithm can be used for reconstructing the surface defined by the cover element and, thus, the outer surface of the object based on the shapes and positions of the shape sensing elements. Or, a three-dimensional interpolation algorithm can be used for reconstructing the surface based on the shape and position of a shape sensing element which may be bent such that it runs in loops or spirals within the cover element covering the object. This allows relatively accurately determining the shape and position of the cover element, thereby reliably providing the information about the shape and position of the cover element and about the position of the first image generating unit relative to the cover element.

In an embodiment the determined shape and position of the cover element defines a surface formed by the cover element, wherein the first image generating unit is adapted to be pressed against the object such that the object is deformed, while generating the first image, wherein the position determination unit is adapted to determine the position of the first image generating unit relative to the cover element by determining the position of a deformation of the reconstructed surface defined by the cover element. This allows determining the position of the first image generating unit relative to the cover element without necessarily needing a further localization device.

In a further embodiment the image generation apparatus comprises markers attached to the flexible cover element and a marker position determination device for determining the positions of the markers, wherein the position determination unit is adapted to determine the position of the first image generating unit relative to the cover element depending on the determined positions of the markers. The markers can be directly attached to the cover element, or indirectly, i.e., for instance, the cover element can be held in a holding device like a bra, wherein the markers can be attached to the bra for indirectly attaching the markers to the cover element. The marker position determination device can be an optical stereo camera system and the markers can be optical markers which are easily recognizable by the stereo camera system.

Preferentially, the image generation apparatus is adapted to image a breast of a woman, wherein the image generation apparatus comprises a holding device for holding the cover element on the breast. The holding device is preferentially a bra, wherein the cover element is integrated in the bra. The cover element can therefore be arranged at a desired position and held in this position in a relatively simple way by using the bra with the integrated cover element.

The first image generating unit is preferentially an ultrasound unit for generating an ultrasound image as the first image, in particular, a hand-held ultrasound device. Thus, even if the first image generating unit is a hand-held ultrasound device like a hand-held ultrasound head, wherein the position of the ultrasound device with respect to the deformable object can vary, the deformation, i.e. the shape and position of the cover element, and the position of the ultrasound device with respect to the cover element can reliably be determined and provided for reviewing purposes, in particular, for registration purposes.

In a further aspect of the present invention a holding device for holding a woman's breast is presented, wherein the holding device comprises a flexible cover element, which includes a shape sensing element for generating a signal depending on its shape, wherein the holding device is adapted such that the flexible cover element covers the breast, if the holding device is worn by the woman.

In a further aspect of the present invention a registration device is presented, wherein the registration device is adapted to register a first image of an object provided by an image generation apparatus as defined in claim 1 and a second image of the object with respect to each other and wherein the registration device comprises:
a surface determination unit for determining a surface of the object in the second image,
a registration unit for registering the first image and the second image with respect to each other based on a shape and position of a cover element covering the object during the generation of the first image provided by the image generation apparatus, a position of a first image generating unit for generating the first image relative to the cover element during the generation of the first image provided by the image generation apparatus, and the determined surface of the object in the second image.

In a further aspect of the present invention an image generation method for generating an image of a deformable object is presented, wherein the image generation method comprises:
covering the object by a flexible cover element,
generating a first image of the object by a first image generating unit,
determining the shape and position of the cover element, the determined shape and position of the cover element being the shape and position during the generation of the first image,
determining the position of the first image generating unit relative to the cover element by a position determination unit, the determined position of the first image generating unit relative to the cover element being the position during the generation of the first image of the object.

In a further aspect of the present invention a registration method is presented, wherein the registration method registers a first image of an object provided by an image generation apparatus as defined in claim 1 and a second image of the object with respect to each other, and wherein the registration method comprises:
determining a surface of the object in the second image by a surface determination unit,
registering the first image and the second image with respect to each other based on a shape and position of a cover element covering the object during the generation of the first image provided by the image generation apparatus, a position of a first image generating unit for generating the first image relative to the cover element during the generation of the first image provided by the image generation apparatus, and the determined surface of the object in the second image by a registration unit.

For registering the first image and the second image also the respective image content, in particular, the image values, which are preferentially gray values, can be used. In a further aspect of the present invention an image generation computer program for generating an image of a deformable object is presented, wherein the image generation computer program comprises program code means for causing an image generation apparatus as defined in claim 1 to carry out the steps of the image generation method as defined in claim 12, when the image generation computer program is run on a computer controlling the image generation apparatus.

In a further aspect of the present invention a registration computer program is presented, wherein the registration computer program comprises program code means for causing a registration device as defined in claim 11 to carry out the steps of the registration method as defined in claim 13, when the registration computer program is run on a computer controlling the registration device.

It shall be understood that the image generation apparatus of claim 1, the holding device of claim 10, the registration device of claim 11, the image generation method of claim 12, the registration method of claim 13, the image generation computer program of claim 14, and the registration computer program of claim 15 have similar and/or identical preferred embodiments, in particular, as defined in the dependent claims.

It shall be understood that a preferred embodiment of the invention can also be any combination of the dependent claims with the respective independent claim.

These and other aspects of the invention will be apparent from and elucidated with reference to the embodiments described hereinafter.

### BRIEF DESCRIPTION OF THE DRAWINGS

In the following drawings:

FIG. 1 shows schematically and exemplarily an embodiment of an image generation apparatus for generating an image of a deformable object,

FIG. 2 shows schematically and exemplarily an embodiment of a membrane of the image generation apparatus,

FIG. 3 shows schematically and exemplarily a bra-like holding structure for holding the membrane such that it covers a woman's breast,

FIGS. 4 and 5 show schematically and exemplarily further embodiments of the membrane, and

FIG. 6 shows a flowchart exemplarily illustrating an embodiment of an image generation method for generating an image of a deformable object.

### DETAILED DESCRIPTION OF EMBODIMENTS

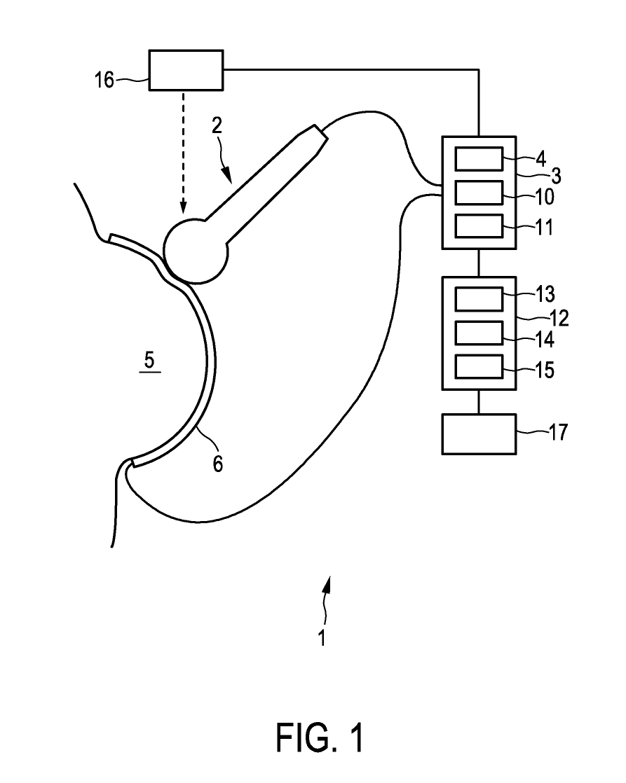

FIG. 1 shows schematically and exemplarily an embodiment of an image generation apparatus for generating an image of a deformable object. In this embodiment the deformable object is a breast 5 of a woman. The image generation apparatus 1 comprises a first image generating unit 2 for generating a first image of the object 5 being an ultrasound head, which is connected with an ultrasound control unit 4 located in a rack 3. The ultrasound head 2 and the ultrasound control unit 4 can be adapted to generate ultrasound images, for instance, spatially two-dimensional or three-dimensional ultrasound images, which may be still images or cine mode images.

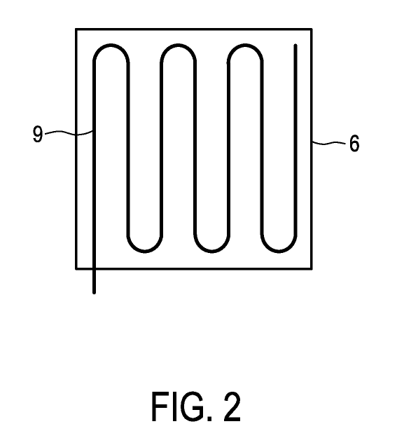

The imaging apparatus 1 further comprises a cover element being, in this embodiment, a flexible membrane 6 covering the object 5, wherein the flexible membrane 6 comprises an optical fiber for FOSSL as a shape sensing element. The arrangement of the optical fiber within the membrane 6 is schematically and exemplarily illustrated in FIG. 2. As can be seen in FIG. 2, the optical fiber 9 may be bent such that it runs in loops or spirals within the membrane 6 covering the object 5.

The image generation apparatus 1 further comprises a shape sensing and localization unit being a fiber-optic shape sensing and localization unit 10, which is connected to the optical fiber 9 and which is adapted to determine the shape and position of the membrane 6 by FOSSL using optical signals from the optical fiber 9. The determined shape and position of the membrane 6 is the shape and position during the generation of the ultrasound image of the object 5. The fiber-optic shape sensing and localization unit 10 is adapted to determine the shape and the position of the membrane 6 by determining the shape and the position of the optical fiber 9 of the membrane 6 and by reconstructing a surface defined by the membrane 6 based on the determined shape and position of the optical fiber 9. For reconstructing the surface preferentially a three-dimensional interpolation algorithm is used for interpolating between the different portions of the optical fiber 9, of which the positions and shapes have been determined.

The image generation apparatus 1 further comprises a position determination unit 11 for determining the position of the ultrasound head 2 relative to the membrane 6. The determined position of the ultrasound head 2 relative to the membrane 6 is the position during the generation of the ultrasound image of the object 5.

The ultrasound head 2 is a hand-held ultrasound head, which is adapted to be pressed against the object 5 such that the object is deformed, while generating the ultrasound image. The position determination unit 11 can be adapted to determine the position of the ultrasound head 2 relative to the membrane 6 by determining the position of a deformation of the reconstructed surface defined by the membrane 6.

For instance, the position of the deformation and, thus, the position of the ultrasound head 2 relative to the membrane 6 can be determined by comparing the reconstructed surface representing the membrane, while the membrane is deformed, with a corresponding surface of the membrane, before deforming the membrane. Alternatively or in addition, a shape template can be provided, wherein the shape template corresponds to the shape of the tip of the ultrasound head 2 and wherein the shape template can be positioned such that it conforms to the deformation of the reconstructed surface defined by the membrane, in order to determine the position of the ultrasound head relative to the membrane.

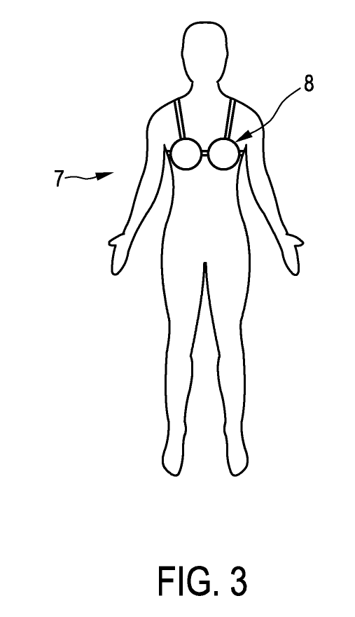

The image generation apparatus can also comprise markers attached to the flexible membrane 6 and a marker position determination device 16 for determining the positions of the markers, wherein the position determination unit 11 can be adapted to determine the position of the ultrasound head 2 relative to the membrane 6 depending on the determined positions of the markers. The markers can be directly attached to the membrane 6, or the markers can be indirectly attached to the membrane 6, i.e., for instance, the membrane 6 can be held in a holding device 8 like a bra worn by a woman 7 as schematically and exemplarily shown in FIG. 3, wherein the markers can be attached to the bra for indirectly attaching the markers to the membrane 6. In this embodiment the marker position determination device 16 is an optical stereo camera system, wherein the markers are optical markers which are easily recognizable by the stereo camera system.

The markers are preferentially directly attached to the membrane 6 or to the bra at positions which substantially do not move during the generation of the first image. For instance, the markers may be arranged on the membrane directly or on the bra such that, if the bra is worn by the woman, the markers are close to the breast or to the chest wall, for example, on the clavicle, on the sternum or on a rip under the breast.

The stereo camera system can record the positions of the markers on the membrane 6 and also record the location and orientation of the ultrasound head 2, which is preferentially also equipped with markers recognizable by the stereo camera system. Preferentially, based on this information the position, i.e. the location and the orientation, of the ultrasound head 2 relative to the membrane 6 at the time of imaging is determined. The marker based determination of the position of the ultrasound head relative to the membrane and the deformation based determination of the position of the ultrasound head relative to the membrane can be combined for improving the accuracy of determining the position of the ultrasound head relative to the membrane. In an embodiment the optical fiber for FOSSL can traverse the markers such that the positions of the markers can also be determined by FOSSL, wherein this additional position information can be used for further improving the accuracy of determining the position of the ultrasound head relative to the membrane.

Since the position of the ultrasound head 2 relative to the membrane 6 may also be determined based on the deformation of the surface defined by the membrane 6, which can be reconstructed based on the determined shape and position of the optical fiber, in an embodiment the image generation apparatus 1 may not comprise the markers and the marker position determination device 16.

In FIG. 1 the membrane 6 is held on the object 5 by using the bra 8, wherein in FIG. 1 elements of the bra 8 are not shown for clarity reasons. The bra 8 is preferentially adapted such that the membrane 6 covers all deformable parts of the breast ranging from the sternum to the axilla and from the clavicle to the inframammary fold.

The image generation apparatus 1 further comprises a registration device 12 for registering the ultrasound image of the object 5 with a second image of the object 5 with respect to each other. The second image is provided by a second image providing unit 13 and is, for instance, a three-dimensional magnetic resonance image of the object. The second image can also be an image of another imaging modality like a computed tomography image or a nuclear image, for example, a positron emission tomography image or a single photon emission computed tomography image. In this embodiment the second image providing unit 13 is a storing unit, in which the already generated second image is stored and from which the second image can be retrieved for providing the same. However, the second image providing unit could also be a receiving unit for receiving the second image from another imaging modality and for providing the received image, or the second image providing unit itself can be an imaging modality for generating and providing the second image.

The registration device 12 further comprises a surface determination unit 14 for determining a surface of the object 5 in the second image. In particular, the surface determination unit 14 is adapted to use known segmentation algorithms for segmenting the surface of the object 5 in the second image. The registration device 12 also comprises a registration unit 15 for registering the ultrasound image and the second image being preferentially a three-dimensional magnetic resonance image with respect to each other based on the shape and position of the membrane 6, which covers the object 5 during the generation of the ultrasound image, based on the position of the ultrasound head 2 relative to the membrane 6 during the generation of the ultrasound image, and based on the determined surface of the object 5 in the second image. In particular, the determined surface of the object 5 in the second image and the reconstructed surface defined by the membrane 6 can be registered with respect to each other, thereby, because the position of the ultrasound head 2 relative to the reconstructed surface defined by the membrane 6 is known, registering the ultrasound image and the second image with respect to each other.

In particular, given the known orientation of the woman during the acquisition of the first image and during the acquisition of the second image, a mapping between locations on the known shape of the membrane 6 and the determined surface of the object in the second image can be established. By this mapping the position of the ultrasound head 2 can be mapped from the first image to the corresponding position on the surface extracted from the second image. Moreover, based on this corresponding position on the surface extracted from the second image the image acquisition geometry used for generating the first image can be simulated on the second image, in order to extract the image section from the second image that corresponds to the first image, thereby registering the ultrasound image and the second image with respect to each other.

The image generation apparatus 1 further comprises a display unit 17 for displaying the first and second images. Since the first and second images are registered with respect to each other, anatomically corresponding locations can be shown in these images.

The membrane 6 is preferentially a polymeric membrane, wherein the optical fiber 9 is interwoven in the membrane 6. The optical fiber can also be included in the membrane in another way. For example, the optical fiber can be included during casting the membrane being, in this case, a foil. The membrane can also comprise two or more foils, wherein the one or several optical fibers can be laminated between the two or more foils. The membrane can also be formed by one or several optical fibers, wherein the one or several optical fibers can form a mesh.

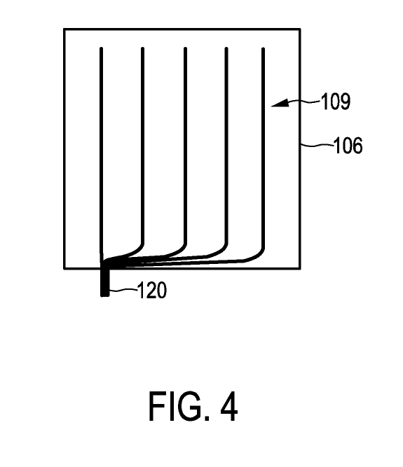

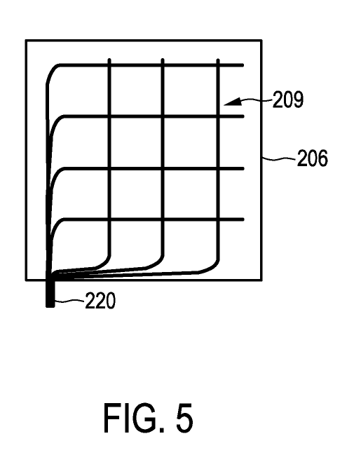

Although in the above described embodiment the membrane 6 only comprises a single optical fiber 9, the membrane can also comprise several optical fibers. For instance, several optical fibers 109 can be arranged in parallel within a membrane 106 with a regular spacing of, for instance, 5 mm, as schematically and exemplarily shown in FIG. 4. Between the membrane 106 and the fiber-optic shape sensing and localization unit 10 the optical fibers 109 form an optical fiber bundle 120 which connects the membrane 106 with the fiber-optic shape sensing and localization unit 10. As schematically and exemplarily shown in FIG. 5, several optical fibers 209 of a membrane 206 can also be arranged in a rectangular grid in the membrane 206, wherein also in this embodiment between the membrane 206 and the fiber-optic shape sensing and localization unit 10 the optical fibers 209 form an optical fiber bundle 220 connecting the membrane 206 with the fiber-optic shape sensing and localization unit 10.

If the membrane comprises several optical fibers, the fiber-optic shape sensing and localization unit 10 is adapted to determine the shape and the position of the several optical fibers of the membrane, i.e. the shape and the position of each optical fiber of the membrane, wherein a surface defined by the membrane is reconstructed based on the determined shapes and positions of the optical fibers, in order to determine the shape and position of the membrane. The reconstruction is preferentially performed by interpolating between the different optical fibers, of which the shapes and positions have been determined.

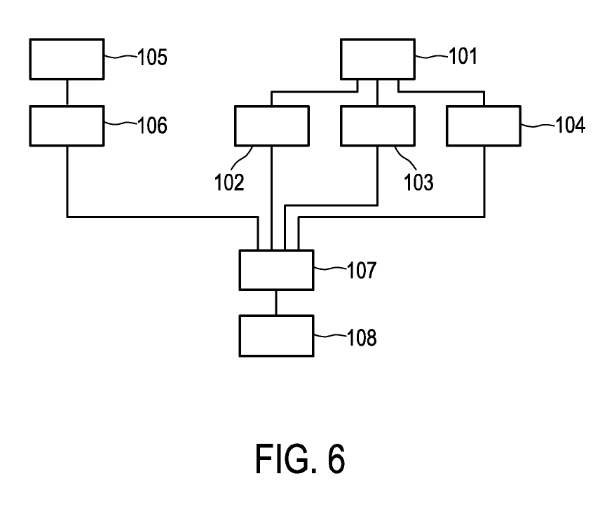

In the following an embodiment of an image generation method for generating an image of a deformable object will exemplarily be described with reference to a flowchart shown in FIG. 6.

In step 301 the object being preferentially a woman's breast is covered by a flexible membrane comprising one or several optical fibers for FOSSL. In particular, a bra-like holding device with the integrated flexible membrane is put on the woman's breast. In step 102 a first image of the deformable object is generated by a first image generating unit. Preferentially, an ultrasound image of the woman's breast is generated by using an ultrasound head. In step 103 the shape and position of the membrane covering the deformable object is determined by FOSSL using the one or several fibers integrated in the membrane by an fiber-optic shape sensing and localization unit connected to the one or several optical fibers. Thus, preferentially the shape and position of the woman's breast and hence the deformation of the woman's breast are determined by FOSSL. In step 104 the position of the first image generating unit relative to the membrane is determined by a position determination unit, in particular, the position of the preferred ultrasound head relative to the membrane covering preferentially the woman's breast is determined.

Steps 102, 103 and 104 are preferentially performed such that the determined shape and position of the membrane is the shape and position of the membrane during the generation of the first image and such that the determined position of the first image generating unit relative to the membrane is the relative position during the generation of the first image of the object.

In step 105 a second image of the object, in particular, a three-dimensional magnetic resonance image, is provided by a second image providing unit, and in step 106 a surface of the object in the second image is determined by a surface determination unit. In step 107 a registration unit registers the first image and the second image with respect to each other based on the determined shape and position of the membrane covering the object, the determined position of the first image generating unit relative to the membrane and the determined surface of the object in the second image. In step 108 the registered first and second images can be shown on a display unit.

Steps 106 to 108 can be regarded as being steps of a registration method for registering first and second images with respect to each other.

In breast cancer diagnosis and treatment planning multiple imaging modalities can be applied, including x-ray mammography, ultrasound imaging and magnetic resonance imaging. For instance, a radiologist may want to detect a lesion that has already been identified on prior magnetic resonance images using a hand-held ultrasound device.

Correlation of breast images between different imaging modalities is generally complicated because of the high degree of deformability of breast tissue. Furthermore, only a limited field of view is generally seen by an ultrasound probe, i.e. an ultrasound head. An automatic registration of, for instance, a two-dimensional ultrasound image to a three-dimensional magnetic resonance image of the breast is therefore generally challenging and error-prone.

The image generation apparatus described above comprises therefore preferentially a FOSSL bra, i.e. a bra comprising a thin, flexible membrane interwoven with one or several optical fibers and equipped with an optic shape sensing system, wherein the FOSSL bra is intended to cover the breast during the ultrasound examination.

The bra can be worn during ultrasound examinations and provides the breast surface shape along with the ultrasound images. By aligning the breast surface shape with the breast shape during other imaging examinations such as three-dimensional breast magnetic resonance imaging, a correlation between the two imaging examinations can be established and a comparison of anatomically corresponding locations can be assured.

Flexible polymeric membranes are used for automated whole breast ultrasound systems. These membranes are ultrasound translucent when covered with acoustic coupling gel. The image generation apparatus described above with reference to, for instance, FIG. 1 uses preferentially a similar membrane, however, in contrast to the known membranes, interwoven with one or several optical fibers. Each fiber is attached to an optical shape sensing system, i.e. to a fiber-optic shape sensing and localization unit, that allows to reconstruct the shape of the respective optical fiber, if the membrane and the inserted fibers are deformed.

The membrane is preferentially inserted in a bra-like device, i.e. in the holding device, that allows the woman to wear the membrane over the breast, while the membrane covers the breast and is in contact with the breast surface throughout. Furthermore, the membrane will be held in place throughout the ultrasound procedure. Before the ultrasound examination is performed, the membrane may be covered with an acoustic gel both on the side of the breast and the outside facing side that will be in contact with the ultrasound transducer of the ultrasound head. Along with the ultrasound images, which may be two-dimensional ultrasound images like two-dimensional ultrasound still images or two-dimensional ultrasound cine mode images or which may be three-dimensional ultrasound images, the optical fiber shapes are preferentially recorded synchronized with the image acquisition such that for each given ultrasound image the fiber shapes are known. From the shapes of the optical fibers a close surface can be reconstructed by three-dimensional interpolation algorithms. Since the ultrasound head, in particular, a hand-held ultrasound transducer, is put on the breast with some pressure, the transducer position relative to the reconstructed breast surface can automatically be recovered from the reconstructed optical fiber surface. Alternatively the relative position of the ultrasound transducer and the breast-covering membrane can be estimated using an optical stereo camera system. For this the FOSSL bra can be equipped with markers visible by the stereo camera system. It is therefore possible to record the current state of the breast shape at the time of the acquisition of an image of the breast.

As a result of this procedure ultrasound images can be provided with known relative position and orientation to the breast surface, as well as the reconstructed breast surface of the whole breast. Therefore, this information, i.e., for instance, the position and orientation of the ultrasound probe, in particular, of the ultrasound transducer, can be stored along with the corresponding ultrasound image for later reference. In a further step these data can be used, in order to correlate the acquired ultrasound images with, for instance, given three-dimensional images of the breast, which may be acquired by using a magnetic resonance imaging scanner. From the given three-dimensional images of the breast the breast surface can directly be reconstructed due to the three-dimensional nature of the imaging procedure. Surface registration algorithms can be used to align the surfaces acquired during both examinations and also to extrapolate the estimated deformation from the surface into the breast tissue. This three-dimensional deformation information allows to transform the ultrasound image into the coordinate system and patient geometry of the three-dimensional breast acquisition, in particular, of the three-dimensional breast magnetic resonance acquisition, which further allows a comparison of the tissue appearance in the ultrasound image and in the further three-dimensional image at anatomically corresponding positions.

The cover element, in particular, the membrane, can preferentially have any shape, which allows the cover element to cover a woman's breast, in particular, to cover all deformable parts of a woman's breast.

Although in the above described embodiments the cover element comprises one or several FOSSL fibers as shape sensing elements, in other embodiments the cover element can also comprise one or several other shape sensing elements like strain gauge elements or other elements, which provide a signal depending on their shape.

Although in an above described embodiment a stereo camera system is used together with markers for determining the position of a first image generating unit relative to the cover element, in other embodiments also other measurement units can be used for measuring the position of, for instance, an ultrasound head relative to the cover element like electromagnetic tracking systems, laser range scanners, structured light positioning systems, ultrasound positioning systems, et cetera.

Although in the embodiment described above with reference to FIG. 1 some units like the ultrasound control unit 4 are arranged in a rack 3, in other embodiments these units may not be arranged in a rack. Moreover, although in the above described embodiments the object to be imaged is preferentially a woman's breast, in other embodiments the image generation apparatus can also be adapted to image another deformable object like an abdominal region of a person or a technical deformable object.

Although in above described embodiments the first image generating unit is adapted to generate an ultrasound image, the first image generating unit can also be adapted to generate another kind of image like an x-ray projection image. The image generation apparatus can be adapted to generate an image of a first imaging modality, wherein the image of the first imaging modality can be registered with an image of a second imaging modality.

Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality.

A single unit or device may fulfill the functions of several items recited in the claims. The mere fact that certain measures are recited in mutually different dependent claims does not indicate that a combination of these measures cannot be used to advantage.

Determinations like the determination of the shape and position of the membrane and the determination of the position of the first image generating unit relative to the membrane, registration procedures, et cetera performed by one or several units or devices can be performed by any other number of units or devices. For example, steps 106 and 107 can be performed by a single unit or by any other number of different units. The determinations, registrations, et cetera and/or the control of the image generation apparatus in accordance with the image generation method and/or the control of the registration device in accordance with the registration method can be implemented as program code means of a computer program and/or as dedicated hardware.

A computer program may be stored/distributed on a suitable medium, such as an optical storage medium or a solid-state medium, supplied together with or as part of other hardware, but may also be distributed in other forms, such as via the Internet or other wired or wireless telecommunication systems.

Any reference signs in the claims should not be construed as limiting the scope.

The invention relates to an image generation apparatus for generating an image of a deformable object like a woman's breast. A first image generating unit like an ultrasound head generates a first image of the object covered by a flexible cover element, wherein a shape sensing and localization unit, in particular, a fiber-optic shape sensing and localization unit, determines the shape and position of the cover element. The apparatus further comprises a position determination unit for determining the position of the first image generating unit relative to the cover element. Information about the deformation of the deformable object with respect to the first image can therefore be provided, which can facilitate reviewing the first image, especially reviewing of breast images. For instance, this information can facilitate a comparison of breast images acquired by different imaging modalities.

1. An image generation apparatus for generating an image of a deformable object, the image generation apparatus comprising:
a first image generating unit for generating a first image of the object,
a flexible cover element for covering the object,
a shape sensing and localization unit for determining the shape and position of the cover element, wherein the determined shape and position of the cover element are the shape and position during the generation of the first image,
a position determination unit for determining the position of the first image generating unit relative to the cover element, wherein the determined position of the first image generating unit relative to the cover element is the position during the generation of the first image of the object.

2. The image generation apparatus as defined in claim 1, wherein the flexible cover element comprises a shape sensing element for generating a signal depending on its shape and wherein the shape sensing and localization unit is connected to the shape sensing element and adapted to determine the shape and position of the cover element based on the signal from the shape sensing element.

3. The image generation apparatus as defined in claim 2, wherein the shape sensing element is an optical fiber for fiber-optic shape sensing and localization and wherein the shape sensing and localization unit is a fiber-optic shape sensing and localization unit being connected to the optical fiber for receiving an optical signal from the optical fiber and being adapted to determine the shape and position of the cover element by fiber-optic shape sensing and localization from the optical signal using the optical fiber.

4. The image generation apparatus as defined in claim 2, wherein the cover element comprises several shape sensing elements which are arranged in parallel or in a rectangular grid in the cover element.

5. The image generation apparatus as defined in claim 2, wherein the shape sensing and localization unit is adapted to determine the shape and the position of the cover element by:
determining the shape and the position of the shape sensing element of the cover element, and
reconstructing a surface defined by the cover element based on the determined shape and position of the shape sensing element, thereby determining the shape and position of the cover element.

6. The image generation apparatus as defined in claim 1, wherein the image generation apparatus further comprises:
a second image providing unit for providing a second image of the object,
a surface determination unit for determining a surface of the object in the second image,
a registration unit for registering the first image and the second image with respect to each other based on the determined shape and position of the cover element covering the object, the determined position of the first image generating unit relative to the cover element and the determined surface of the object in the second image.

7. The image generation apparatus as defined in claim 1, wherein the determined shape and position of the cover element defines a surface formed by the cover element, wherein the first image generating unit is adapted to be pressed against the object such that the object is deformed, while generating the first image, wherein the position determination unit is adapted to determine the position of the first image generating unit relative to the cover element by determining the position of a deformation of the surface defined by the cover element.

8. The image generation apparatus as defined in claim 1, wherein the image generation apparatus further comprises markers attached to the flexible cover element and a marker position determination device for determining the positions of the markers, wherein the position determination unit is adapted to determine the position of the first image generating unit relative to the cover element depending on the determined positions of the markers.

9. The image generation apparatus as defined in claim 1, wherein the image generation apparatus is adapted to image a breast of a woman, wherein the image generation apparatus further comprises a holding device for holding the cover element on the breast.

10. A holding device for holding a woman's breast, wherein the holding device comprises a flexible cover element, which includes a shape sensing element for generating a signal depending on its shape, wherein the holding device is adapted such that the flexible cover element covers the breast, if the holding device is worn by the woman.

11. A registration device, wherein the registration device is adapted to register a first image of an object provided by an image generation apparatus as defined in claim 1 and a second image of the object with respect to each other, and wherein the registration device comprises:
a surface determination unit for determining a surface of the object in the second image,
a registration unit for registering the first image and the second image with respect to each other based on a shape and position of a cover element covering the object during the generation of the first image provided by the image generation apparatus, a position of a first image generating unit for generating the first image relative to the cover element during the generation of the first image provided by the image generation apparatus, and the determined surface of the object in the second image.

12. An image generation method for generating an image of a deformable object, the image generation method comprising:
covering the object by a flexible cover element,
generating a first image of the object by a first image generating unit,
determining the shape and position of the cover element, the determined shape and position of the cover element being the shape and position during the generation of the first image,
determining the position of the first image generating unit relative to the cover element by a position determination unit, the determined position of the first image generating unit relative to the cover element being the position during the generation of the first image of the object.

13. A registration method, wherein the registration method registers a first image of an object provided by an image generation apparatus as defined in claim 1 and a second image of the object with respect to each other, and wherein the registration method comprises:
determining a surface of the object in the second image by a surface determination unit,
registering the first image and the second image with respect to each other based on a shape and position of a cover element covering the object during the generation of the first image provided by the image generation apparatus, a position of a first image generating unit for generating the first image relative to the cover element during the generation of the first image provided by the image generation apparatus, and the determined surface of the object in the second image by a registration unit.

14. An image generation computer program for generating an image of a deformable object, the image generation computer program comprising program code means for causing an image generation apparatus to carry out the steps of the image generation method as defined in claim 12, when the image generation computer program is run on a computer controlling the image generation apparatus.

15. A registration computer program comprising program code means for causing a registration device to carry out the steps of the registration method as defined in claim 13, when the registration computer program is run on a computer controlling the registration device.
